(19) United States
(12) Patent Application Publication
(10) Pub. No.: US 2013/0110094 A1
(43) Pub. Date: May 2, 2013

(54) Patient transfer arrangement, medical treatment apparatus and medical treatment system

(71) Applicants: Armin Wellhöfer, Schwaig (DE); Christof Donitzky, Eckental (DE)

(72) Inventors: Armin Wellhöfer, Schwaig (DE); Christof Donitzky, Eckental (DE)

(21) Appl. No.: 13/282,022

(22) Filed: Oct. 26, 2011

(51) Int. Cl. A61G 7/10; A61B 18/20

(52) U.S. Cl. 606/5; 5/81.1 R

(57) ABSTRACT

A patient transfer arrangement includes a movable substructure and a carrier member that is connected to the substructure and that is adapted to receive a patient in a sitting or lying position. A connecting device is detachably connectable to a complementary connecting device provided on a base unit of a medical treatment apparatus, in order to fasten the carrier member detachably to the medical treatment apparatus in such a position that a patient supported on the carrier member in a sitting or lying position is accessible to a medical treatment by means of a treatment unit of the medical treatment apparatus.

The invention relates to a patient transfer arrangement, to a medical treatment apparatus that is suitable for interacting with a patient transfer arrangement of such a type, and also to a medical treatment system comprising a patient transfer arrangement of such a type and a medical treatment apparatus of such a type.

### BACKGROUND OF THE INVENTION

In the course of the implementation of medical treatments it is frequently necessary to transfer the patient to be treated to another bed. For example, it may be necessary to transport the patient in a sitting or lying position to a medical treatment apparatus and subsequently to transfer him/her onto a seat assigned to the medical treatment apparatus or onto a couch assigned to the medical treatment apparatus. The transfer of the patient to another bed may be taxing, disagreeable and even associated with an increased risk of accidents, both for the nursing staff and for the patient. Moreover, two seats or couches, namely a mobile arrangement and an immobile arrangement assigned to the medical treatment apparatus, are always required.

### BRIEF SUMMARY OF THE INVENTION

The object underlying the invention is to make a patient transfer arrangement available that enables the transportation of a patient to a medical treatment apparatus and that enables the medical treatment of the patient by means of the medical treatment apparatus without it being necessary during these procedures to transfer the patient to another bed. Furthermore, the invention is directed towards the object of specifying a medical treatment apparatus that is suitable for interacting with a patient-treatment arrangement of such a type and also a medical treatment system that comprises a patient transfer arrangement of such a type and a medical treatment apparatus of such a type.

This object is achieved by a patient transfer arrangement with the features of Claim 1, by a medical treatment apparatus with the features of Claim 9, and by a medical treatment system with the features of Claim 16.

The patient transfer arrangement according to the invention includes a movable substructure. The movable substructure may be, for example, a frame provided with a plurality of wheels or castors. Connected to the substructure is a carrier member that is adapted to receive a patient in a sitting or lying position. The carrier member consequently constitutes a seat surface or couch surface for a patient. Lastly, the patient transfer arrangement includes a connecting device that is detachably connectable to a complementary connecting device provided on a base unit of a medical treatment apparatus. By virtue of the interaction of the connecting device of the patient transfer arrangement and the connecting device of the medical treatment apparatus, the carrier member is detachably fastened to the medical treatment apparatus in such a position that a patient supported on the carrier member in a sitting or lying position is accessible to a medical treatment by means of a treatment unit of the medical treatment apparatus. The medical treatment apparatus may be a diagnostic or therapeutic apparatus. The treatment unit of the medical treatment apparatus may consequently be designed in the form of a diagnostic unit or a therapeutic unit. The treatment unit may serve to carry out surgical interventions, in particular refractive surgical interventions on the human eye.

By virtue of the equipping of the patient transfer arrangement according to the invention with a connecting device that is suitable for interacting with a complementary connecting device provided on the base unit of the medical treatment apparatus, the patient transfer arrangement can be fastened in simple manner to the medical treatment apparatus in such a way that the patient can be transported to the medical treatment apparatus and can be subjected to a medical treatment by means of the treatment unit of the medical treatment apparatus without a transfer of the patient to another bed being required for this purpose. Inconvenience for the nursing staff and the patient that is associated with the transfer to another bed may consequently be avoided by virtue of the patient transfer arrangement according to the invention. As a result, it becomes possible, for example, to prepare for the medical treatment a patient supported on the patient transfer arrangement at a place remote from the medical treatment apparatus, for example in a preparation room or the like, and subsequently to bring him/her quickly and without difficulty into a position relative to the medical treatment apparatus in which the patient can be treated by means of the treatment unit of the medical treatment apparatus. Moreover, the medical treatment apparatus does not require an additional carrier member for supporting the patient. The space requirement of the medical treatment apparatus becomes smaller as a result. Moreover, space for the accommodation of the patient transfer arrangement during the treatment of the patient by means of the treatment unit of the medical treatment apparatus can be saved.

The connecting device of the patient transfer arrangement is adapted to interact with the complementary connecting device of the medical treatment apparatus in such a manner that the patient transfer arrangement is reproducibly connected in the same position to the medical treatment apparatus or, to be more exact, to the base unit of the medical treatment apparatus. As a result, the home position of the patient transfer arrangement is defined relative to the medical treatment apparatus or, to be more exact, relative to the medical treatment unit of the medical treatment apparatus. As a result, elaborate measures for correct positioning of the patient transfer arrangement relative to the treatment unit of the medical treatment apparatus can be dispensed with. This not only enhances the treatment comfort for the nursing staff, the physician and the patient but also improves the safety of the treatment and the work flow. This is particularly advantageous when the patient transfer arrangement according to the invention is being utilised for application with a medical treatment apparatus with which many patients are treated within a short time.

By virtue of the interaction of the connecting device of the patient transfer arrangement and the connecting device of the medical treatment apparatus, the carrier member of the patient transfer arrangement according to the invention may be fastenable, jointly with the substructure, to the medical treatment apparatus, in particular to the base unit of the medical treatment apparatus. Given such a configuration of the patient transfer arrangement, the patient transfer arrangement can be fastened as a whole to the medical treatment apparatus.

Alternatively, the carrier member of the patient transfer arrangement according to the invention may also be detachably connected to the substructure of the patient transfer arrangement and, by virtue of the interaction of the connecting device of the patient transfer arrangement and the connecting device of the medical treatment apparatus, may be fastenable to the medical treatment apparatus in a state detached from the substructure. Given such a configuration of the patient transfer arrangement, the carrier member may, for example, be detached from the substructure in a first step and subsequently, by virtue of the interaction of the connecting devices of the patient transfer arrangement and of the medical treatment apparatus, may be fastened to the medical treatment apparatus separately from the substructure for the duration of the medical treatment of the patient supported on the carrier member. After conclusion of the medical treatment, the carrier member can again be detached from the medical treatment apparatus and connected once more to the substructure of the patient transfer arrangement.

Irrespective of whether the patient transfer arrangement as a whole or only the carrier member of the patient transfer arrangement is fastened to the medical treatment apparatus, the height of the substructure is adapted to the height of the base unit of the medical treatment apparatus, in order to enable a comfortable displacement of the patient transfer arrangement or of the carrier member onto the base unit of the medical treatment apparatus and a simple fastening of the patient transfer arrangement or of the carrier member on the base unit of the medical treatment apparatus.

The patient transfer arrangement according to the invention may include a guiding device which includes a guiding element fastened to the carrier member and a guiding element fastened to the substructure and complementary to the guiding element of the carrier member. The guiding device is preferentially adapted to enable, by virtue of the interaction of the guiding element of the carrier member and the guiding element of the substructure, a guided motion of the carrier member relative to the substructure when the carrier member is being detached from the substructure. The guiding elements of the guiding device may, for example, be designed in the form of complementary rails which preferentially enable a displacement of the carrier member relative to the substructure in a direction substantially perpendicular to a longitudinal axis of the patient transfer arrangement.

A guiding element provided on the patient transfer arrangement may be adapted to interact with a complementary guiding element provided on the medical treatment apparatus, in order to enable, by virtue of the interaction of the guiding element of the patient transfer arrangement and the guiding element of the medical treatment apparatus, a guided motion of the patient transfer arrangement relative to the medical treatment apparatus. If the patient transfer arrangement according to the invention is designed in such a way that the carrier member can be detached from the substructure and connected to the medical treatment apparatus, the guiding element of the carrier member may serve as a guiding element of the patient transfer arrangement which is provided for interacting with the guiding element of the medical treatment apparatus, said guiding element of the carrier member serving, in the state of the carrier member connected to the substructure, for guiding a relative motion between the carrier member and the substructure. The guiding element of the medical treatment apparatus may then, for example, be constructed in the form of a rail that is similar to the guiding element of the substructure.

The patient transfer arrangement according to the invention may further include a coupling device that is connectable to a positioning system provided on the medical treatment apparatus for varying the position of the carrier member of the patient transfer arrangement relative to the medical treatment apparatus. The coupling device may, for example, be configured in such a way that it can connect the patient transfer arrangement to a drive unit of the positioning system. The coupling device consequently enables a functional connection of the patient transfer arrangement to the medical treatment apparatus. The positioning system may be adapted to vary the position of the carrier member relative to the medical treatment apparatus in the x-, y- and/or z-direction.

The carrier member of the patient transfer arrangement according to the invention may be adjustable between a first operating position, in which it enables a support of a patient in a sitting position, and a second operating position, in which it enables a support of a patient in a lying position. The patient can then, for example in a sitting position, be prepared for the medical treatment by means of the treatment unit of the medical treatment apparatus and can be transported to the medical treatment apparatus. With a view to the treatment itself, the patient can then be brought into a lying position without a transfer of the patient to another bed being required for this purpose. The substructure of a patient transfer arrangement, the carrier member of which is adjustable between two operating positions, is preferentially likewise adjustable between a first operating position and a second operating position. In its first operating position the substructure may exhibit a lower height than in its second operating position. As a result, in each operating position of the carrier member a comfortable support of a patient on the carrier member is made possible.

The patient transfer arrangement according to the invention may further include at least one holding device for fastening a medical support device to the patient transfer arrangement. The medical support device may be, for example, an electrocardiograph, a monitor, a drug-dosing apparatus, an oxygen-supply apparatus or the like. Since the patient transfer arrangement according to the invention renders a transfer of the patient to another bed superfluous, the patient can be coupled to the medical support device already within the scope of the preparation for the medical treatment and subsequently, jointly with at least the carrier member of the patient transfer arrangement and with the medical support device, can be brought into a position in which the patient is accessible to the medical treatment by means of the treatment unit of the medical treatment apparatus.

A medical treatment apparatus according to the invention includes a treatment unit which may be constructed in the form of a medical diagnostic and/or therapeutic unit. The medical treatment apparatus further includes a base unit with a connecting device that is connectable to a complementary connecting device provided on a patient transfer arrangement, in order to fasten a carrier member of the patient transfer arrangement detachably to the medical treatment apparatus in such a position that a patient supported on the carrier member in a sitting or lying position is accessible to a medical treatment by means of the treatment unit of the medical treatment apparatus.

The medical treatment apparatus is preferably configured in such a way that, by virtue of the interaction of the connecting device of the patient transfer arrangement and the connecting device of the medical treatment apparatus, the carrier member of the patient transfer arrangement is fastenable to the medical treatment apparatus jointly with a movable substructure of the patient transfer arrangement. The medical treatment apparatus should then be provided with a base unit that does not impede a fastening of the patient transfer arrangement with the movable substructure to the medical treatment apparatus.

The medical treatment apparatus may, however, also be configured in such a way that, by virtue of the interaction of the connecting device of the patient transfer arrangement and the connecting device of the medical treatment apparatus, the carrier member of the patient transfer arrangement is fastenable to the medical treatment apparatus in a state detached from the substructure of the patient transfer arrangement. In this case the base unit of the medical treatment apparatus should be capable of supporting the carrier member of the patient transfer arrangement, i.e. of undertaking the supporting function of the travelling substructure of the patient transfer arrangement.

The medical treatment apparatus may include a guiding element that is adapted to interact with a complementary guiding element provided on the patient transfer arrangement, in order to enable, by virtue of the interaction of the guiding element of the medical treatment apparatus and the guiding element of the patient transfer arrangement, a guided motion of the patient transfer arrangement relative to the medical treatment apparatus.

The medical treatment apparatus preferentially includes a positioning system that is connectable to a coupling device of the patient transfer arrangement and that is adapted to vary a position of the carrier member of the patient transfer arrangement relative to the medical treatment apparatus. The positioning system may include a drive unit that permits a variation of the position of the carrier member of the patient transfer arrangement relative to the medical treatment apparatus in the x-, y- and/or z-direction.

Furthermore, the medical treatment apparatus may include a control unit for controlling the positioning system. An operating panel including a keyboard and/or a touch-screen may be assigned to the control unit, via which a user of the medical treatment apparatus can enter positioning commands. The control unit then controls the positioning system in such a manner that the carrier member of the patient transfer arrangement and consequently the patient supported on the carrier member is positioned as desired relative to the medical treatment apparatus and in particular relative to the medical treatment unit of the medical treatment apparatus.

As already mentioned, the treatment unit of the medical treatment apparatus may be constructed in the form of an arbitrary diagnostic and/or therapeutic unit. However, the treatment unit preferentially includes a laser source for carrying out refractive surgical interventions on the human eye. In the case of a medical treatment apparatus, the treatment unit of which serves for carrying out refractive interventions on the human eye, the positioning of the patient relative to the laser source of the treatment unit is of crucial significance. The advantages of a simple, reliable and reproducible positioning of the patient relative to the treatment unit are brought to bear particularly effectively in the case of a medical treatment apparatus of such a type.

A medical treatment system according to the invention includes a patient transfer arrangement, described above, and also a treatment apparatus, described above, that is suitable for interacting with a patient transfer arrangement of such a type.

### BRIEF DESCRIPTION OF THE DRAWINGS

Preferred embodiments of the invention will now be elucidated in greater detail with reference to the appended schematic drawings, in which

FIG. 1 shows a first embodiment of a medical treatment system with a patient transfer arrangement and also with a medical treatment apparatus provided for connection to the patient transfer arrangement,

FIG. 2 shows the medical treatment system according to FIG. 1, wherein, however, the patient transfer arrangement is connected to the medical treatment apparatus,

FIG. 3 shows the medical treatment system according to FIG. 1, wherein, however, the patient transfer arrangement has been brought, by means of a positioning system of the medical treatment apparatus, into a position in which a patient supported on the carrier member of the patient transfer arrangement can be subjected to a medical treatment by means of a medical treatment unit of the medical treatment apparatus,

FIG. 4 shows a second embodiment of a medical treatment system with a patient transfer arrangement and also with a medical treatment apparatus provided for connection to the patient transfer arrangement,

FIG. 5 shows the medical treatment system according to FIG. 4, wherein, however, a carrier member of the patient transfer arrangement is detached from an underframe of the patient transfer arrangement and connected to the medical treatment apparatus,

FIG. 6 shows a further patient transfer arrangement with a carrier member, wherein the carrier member is located in a first operating position which is suitable for supporting a patient on the carrier member in a sitting position,

FIG. 7 shows the patient transfer arrangement according to FIG. 6, wherein, however, the carrier member is located in a second operating position which is suitable for supporting a patient on the carrier member in a lying position, and

FIG. 8 shows the patient transfer arrangement according to FIGS. 6 and 7, wherein the patient transfer arrangement is connected to a medical treatment apparatus.

### DETAILED DESCRIPTION OF PREFERRED EMBODIMENTS

A treatment system 100 shown in the Figures comprises a patient transfer arrangement 10 and also a medical treatment apparatus 12. The patient transfer arrangement 10 has a movable substructure 14 which, in the embodiments of a patient transfer arrangement shown in the Figures, is designed in the form of a frame and includes a plurality of wheels or castors 16. The substructure 14 serves for supporting a carrier member 18.

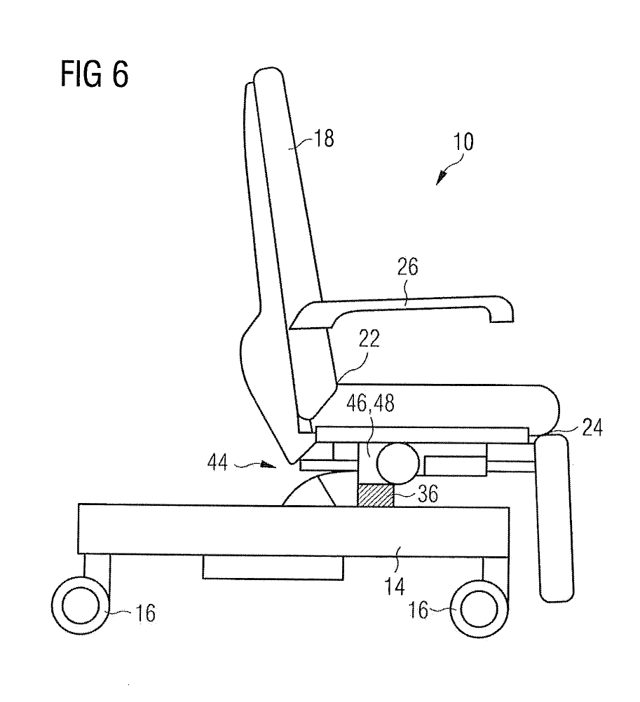

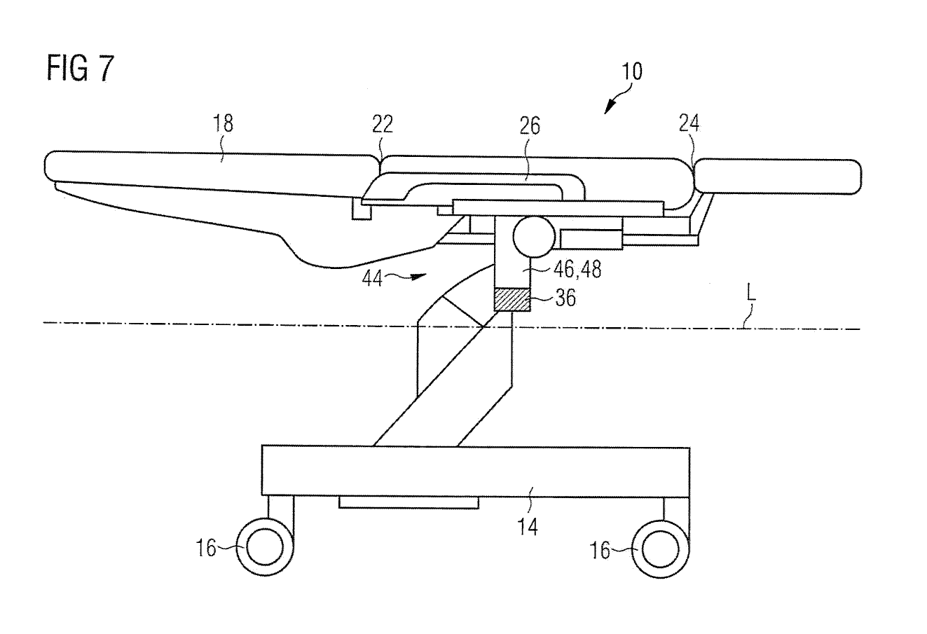

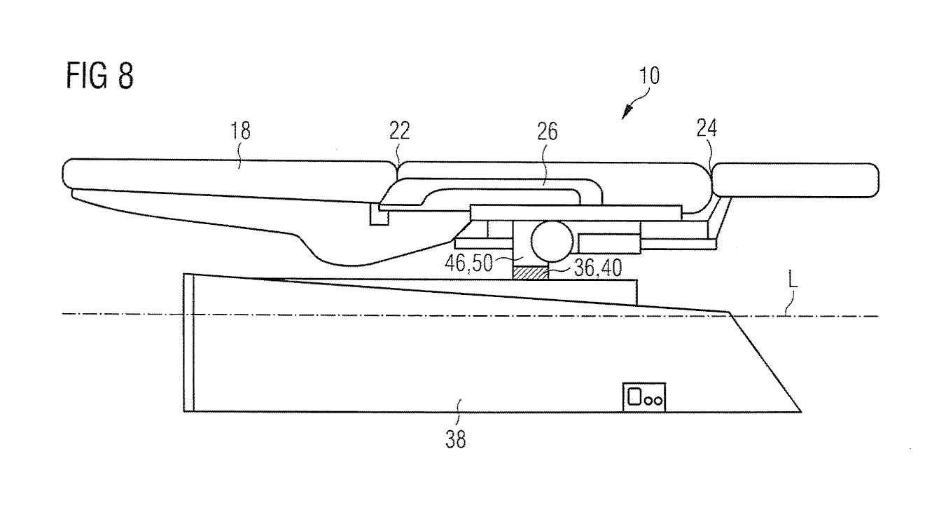

The carrier member 18 of the patient transfer arrangement 10 may in principle be provided merely for supporting a patient 20 in a lying position. Alternatively, the carrier member 18 as illustrated in FIGS. 6 to 8 may, however, also be adjustable between a first operating position (see FIG. 6) and a second operating position (see FIGS. 7 and 8). In its first operating position the carrier member 18 enables the support of a patient in a sitting position, whereas in its second operating position it enables the support of a patient in a lying position. In order to enable an adjustment of the carrier member 18 between its first and its second operating positions, the carrier member 18 is provided with two articulations 22, 24. Moreover, the carrier member exhibits two armrests 26 which can be folded down when the carrier member 18 is located in its second operating position.

In the case of the patient transfer arrangement 10 according to FIGS. 6 to 8, the substructure 14 is furthermore adjustable between a first operating position (see FIG. 6) and a second operating position (see FIG. 7). In its first operating position the substructure 14 exhibits a lower height, in order to enable a comfortable support of a patient 20 on the carrier member 18 when the carrier member 18 is located in its first operating position, in which it serves for supporting a patient 20 in a sitting position. In contrast, in its second operating position the substructure 14 exhibits a greater height, in order to enable a comfortable lying support of a patient on a carrier member 18 located in its second operating position.

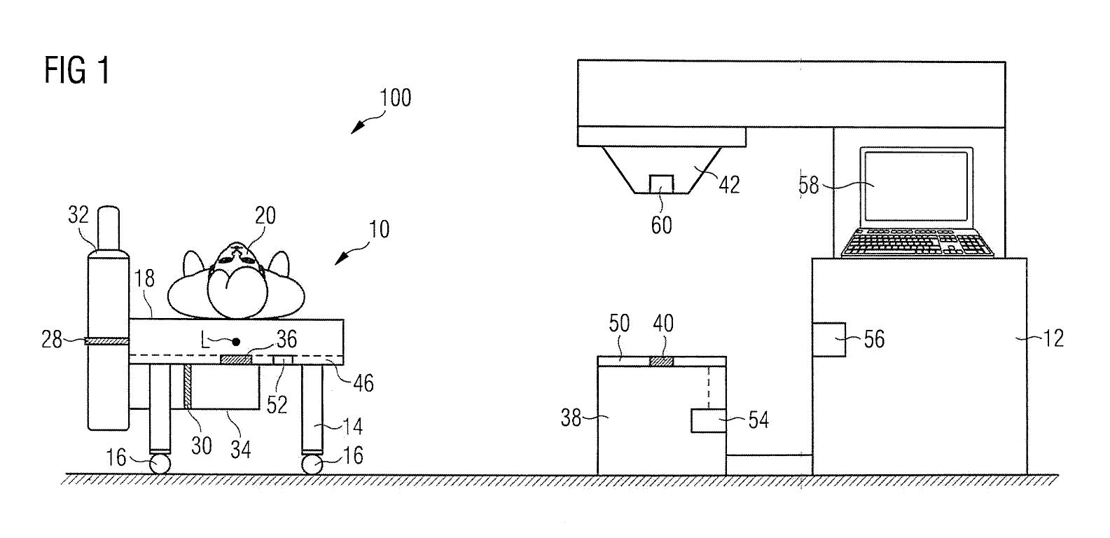

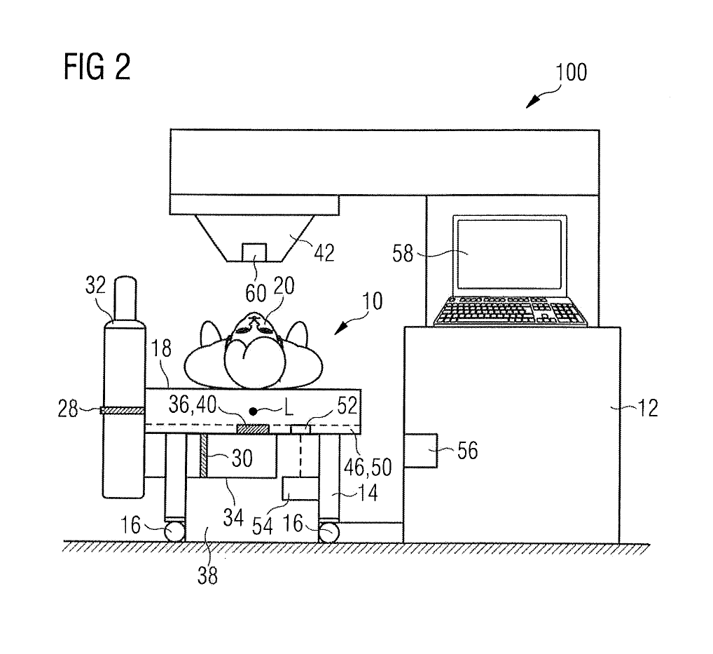

As shown in FIGS. 1 to 5, the patient transfer arrangement 10 further includes holding devices 28, 30 which serve to fasten medical support devices 32, 34 to the patient transfer arrangement 10. In the embodiments shown, the medical support devices 32, 34 are an oxygen-supply system and an electrocardiograph. It will be understood, however, that by means of the holding devices 28, 30 other medical support devices can also be connected to the patient transfer arrangement 10. In the embodiments shown, the holding devices 28, 30 are fastened to the carrier member 18 of the patient transfer arrangement 10. The medical support devices 32, 34 are consequently capable of moving jointly with the carrier member 18 of the patient transfer arrangement 10. In the case of the patient transfer arrangement 10 shown in FIGS. 1 to 3, in which, as will be elucidated in still more detail later, no separation of the carrier member 18 from the substructure 14 takes place, the holding devices 28, 30 could, however, also be mounted on the substructure 14.

The patient transfer arrangement 10 further includes a connecting device 36 which is only indicated schematically in the Figures. The connecting device 36 is provided for the purpose of being detachably connected to a complementary connecting device 40 provided on a base unit 38 of the medical treatment apparatus 12. By virtue of the interaction of the connecting devices 36, 40 the carrier member 18 can be detachably fastened to the medical treatment apparatus 12 in such a position that a patient 20 supported on the carrier member 18 is accessible to a medical treatment by means of a treatment unit 42 of the medical treatment apparatus. The connecting devices may be constructed in the form of special mechanical devices and may include bolts, hooks, clips, a detent mechanism or such like. What is essential is merely that by virtue of the interaction of the connecting devices 36, 40 a stable connection can be established between the carrier member of the patient transfer arrangement 10 and the base unit 38 of the medical treatment apparatus 12. Moreover, by virtue of the construction and the arrangement of the connecting devices 36, 40 it should be ensured that the carrier member 18 is always located in the same position relative to the base unit 38 of the medical treatment apparatus 12 when the connecting device 36 of the patient transfer arrangement 10 is in engagement with the connecting device 40 of the medical treatment apparatus 12.

In the case of the medical treatment system 100 illustrated in FIGS. 1 to 3, the carrier member 18 of the patient transfer arrangement 10 is capable of being fastened to the medical treatment apparatus 12 jointly with the substructure 14 of the patient transfer arrangement 10 by virtue of the interaction of the connecting devices 36, 40. In other words, the patient transfer arrangement 10 can be integrally fastened to the medical treatment apparatus 12. For this purpose the base unit 38 of the medical treatment apparatus 12 has been shaped in such a way that the patient transfer arrangement 10 can be pushed over the base unit 38 in such a way that the base unit 38 can be connected to the carrier member 18 without the substructure 14 of the patient transfer arrangement 10 getting in the way.

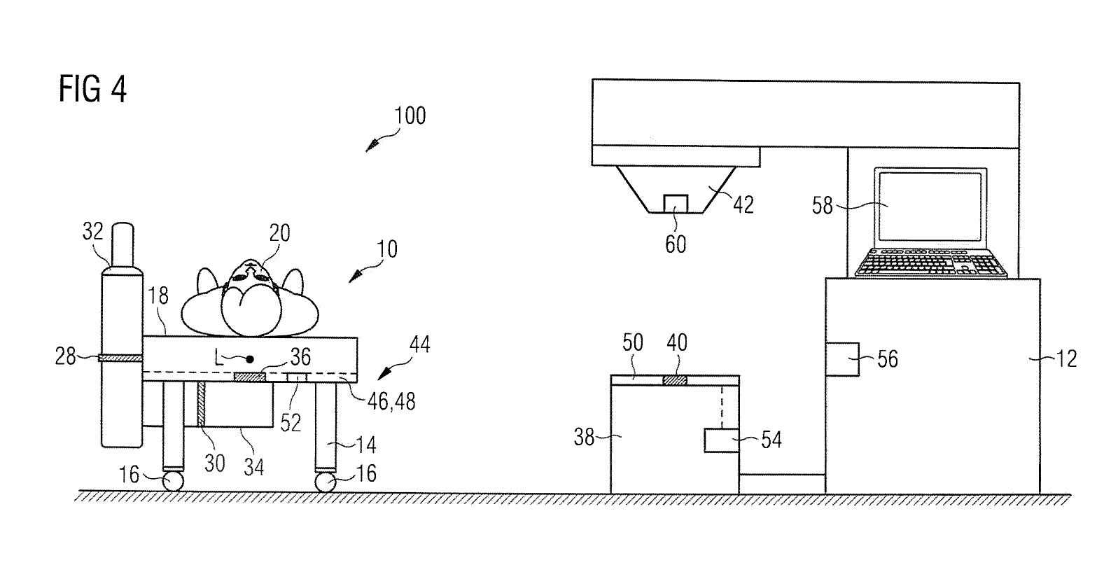

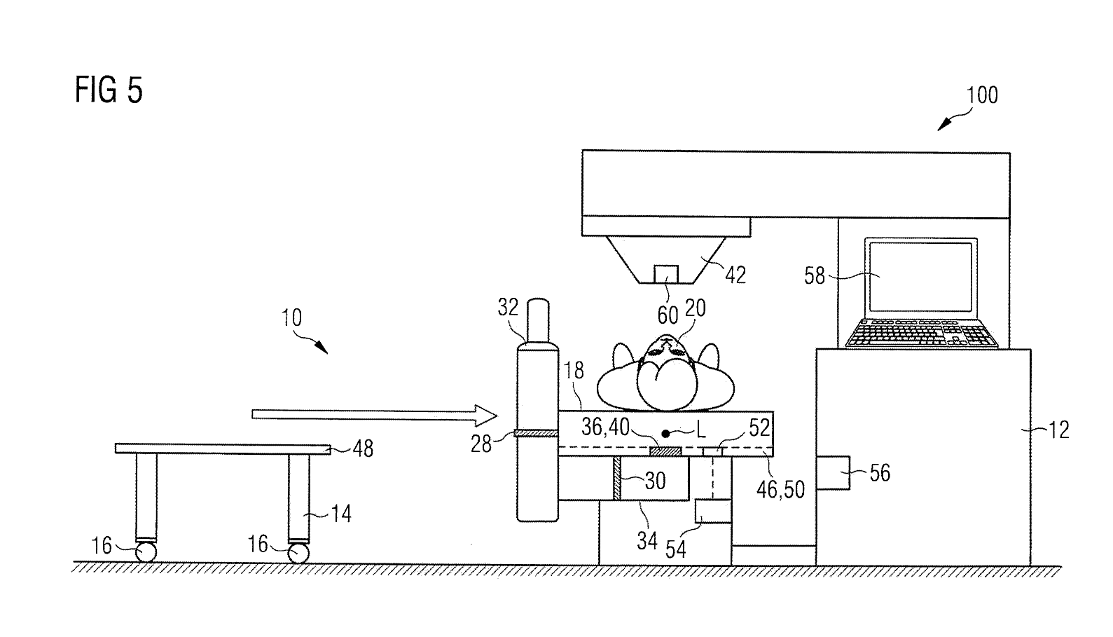

Alternatively, the carrier member 18, as illustrated in FIGS. 4 and 5, may also be detachably connected to the substructure 14. By virtue of the interaction of the connecting devices 36, 40 the carrier member 18 is then capable of being fastened to the medical treatment apparatus 12 in a state detached from the substructure 14. For the purpose of fastening the carrier member 18 to the medical treatment apparatus 12 the carrier member 18 may, for example, be pushed, substantially perpendicular to a longitudinal axis L of the patient transfer arrangement 10, from the substructure 14 onto the base unit 38 of the medical treatment apparatus 12. The base unit 38 then undertakes the supporting function of the substructure 14. In each case the height of the substructure 14 of the patient transfer arrangement 10 is adapted to the height of the base unit 38 of the medical treatment apparatus 12, in order to enable a comfortable displacement of the patient transfer arrangement 10 or, to be more exact, of the carrier member 18 onto the base unit 38 of the medical treatment apparatus 12.

As can best be discerned in FIG. 7, a patient transfer arrangement 10 in which the carrier member 18 has been detachably connected to the substructure 14 includes a guiding device 44 which exhibits a guiding element 46 fastened to the carrier member 18 and also a guiding element 48 fastened to the substructure 14 and complementary to the guiding element 46 of the carrier member 18. In the embodiments shown, the guiding elements 46, 48 are designed in the form of rail elements which in the course of interacting, i.e. when the guiding elements 46, 48 of the guiding device 44 engage with one another, enable a guided motion of the carrier member 18 substantially perpendicular to the longitudinal axis L of the patient transfer arrangement 10 relative to the substructure 14. As a result, the carrier member 18 can be detached from the substructure 14 in controlled manner and can be pushed onto the base unit 38 of the medical treatment apparatus 12.

A guiding element 50 is provided on the base unit 38 of the medical treatment apparatus 12. The guiding element 50 of the medical treatment apparatus 12 serves to interact with a guiding element 46 of the patient transfer arrangement 10, in order to enable a guided displacement of the carrier member 18 of the patient transfer arrangement 10 relative to the base unit 38 of the medical treatment apparatus 12. When the patient transfer arrangement 10, as illustrated in FIGS. 1 to 3, is integrally connected to the medical treatment apparatus 12, the guiding element 50 of the medical treatment apparatus 12 interacts with a guiding element 46 of the patient transfer arrangement 10 which can be fastened to the carrier member 18 of the patient transfer arrangement 10 or to the substructure 14 of the patient transfer arrangement 10.

In the case of a medical treatment apparatus 12 which is provided for interacting with a patient transfer arrangement 10 having a carrier member 18 that is detachably connected to the substructure 14, the structural design of the guiding element 50 corresponds to the structural design of the guiding element 48 of the substructure 14. The guiding element 50 of the base unit 38 can then interact with the guiding element 46 of the carrier member 18 in the same manner as the guiding element 48 of the substructure 14. In the embodiments shown, the guiding elements 46, 50 are designed in the form of rail elements which in the course of interacting, i.e. when the guiding elements 46, 50 engage with one another, enable a guided motion of the patient transfer arrangement 10 or, to be more exact, of the carrier member 18 of the patient transfer arrangement 10 substantially perpendicular to the longitudinal axis L of the patient transfer arrangement 10 relative to the base unit 38 of the medical treatment apparatus 12.

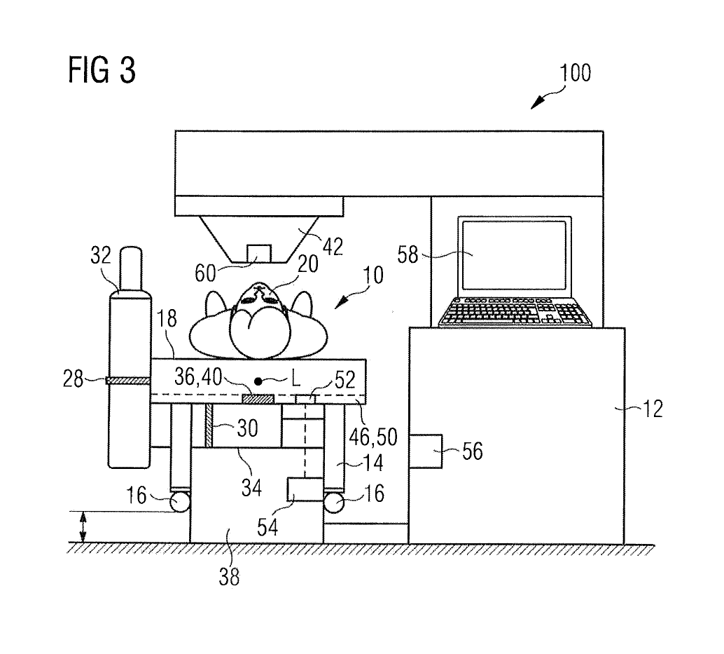

The patient transfer arrangement 10 further includes a coupling device 52 which is only indicated schematically in the Figures. The coupling device 52 serves for coupling the patient transfer arrangement 10 or, to be more exact, the carrier member 18 of the patient transfer arrangement 10 to a positioning system 54 of the medical treatment apparatus 12. The positioning system 54 serves to vary the position of the carrier member 18 relative to the medical treatment apparatus 12 when the carrier member 18 is connected to the medical treatment apparatus 12. The positioning system 54 may include a hydraulic, pneumatic, mechanical or electric drive device which enables a motion of the carrier member 18 relative to the base unit 38 of the medical treatment apparatus 12 in the x-, y- and/or z-direction. In the representation according to FIG. 3 the patient transfer arrangement 10 has been moved by means of the positioning system 54 of the medical treatment apparatus 12 in the z-direction relative to the medical treatment apparatus 12, i.e. raised relative to the base unit 38 of the medical treatment apparatus 12. By means of the positioning system 54, a patient 20 supported on the carrier member 18 can consequently be brought into an optimal position relative to the treatment unit 42 of the medical treatment apparatus 12. However, the positioning system 54 serves substantially for fine adjustment of the position of the patient relative to the treatment unit 42 of the medical treatment apparatus 12, since a rough positioning already takes place by virtue of the positioning of the patient 20 on the carrier member 18 and by virtue of the positioning of the carrier member 18 relative to the medical treatment apparatus 12 by means of the connecting devices 36, 40.

The positioning system 54 is controlled by means of an electronic control unit 56. The electronic control unit 56 can process inputs of a user which the user can enter via an operating panel 58 including, for example, a keyboard. The operating panel 58 also serves the user for the purpose of entering commands for controlling the treatment unit 42. Alternatively, the operating panel may be a computer unit.

In principle, the medical treatment apparatus 12 may be an arbitrary medical diagnostic and/or therapeutic apparatus. In the embodiments of a treatment system 100 that are shown, however, the treatment unit 42 of the medical treatment apparatus 12 includes at least one laser source 60 that is suitable for carrying out refractive surgical interventions on the human eye. In the case of a medical treatment system 100 of such a type the advantage of the system 100, namely that a transfer of the patient 20 to another bed from a patient transfer arrangement 10 onto a carrier member of the medical treatment apparatus 12 can be dispensed with, is brought to bear particularly effectively.

1. Patient transfer arrangement with:
a movable substructure,
a carrier member that is connected to the substructure and that is adapted to receive a patient in a sitting or lying position, and
a connecting device that is detachably connectable to a complementary connecting device provided on a base unit of a medical treatment apparatus, in order to fasten the carrier member detachably to the medical treatment apparatus in such a position that a patient supported on the carrier member in a sitting or lying position is accessible to a medical treatment by means of a treatment unit of the medical treatment apparatus.

2. Patient transfer arrangement according to claim 1,
characterised in that by virtue of the interaction of the connecting device of the patient transfer arrangement and the connecting device of the medical treatment apparatus the carrier member is fastenable to the medical treatment apparatus jointly with the substructure.

3. Patient transfer arrangement according to claim 1,
characterised in that the carrier member is detachably connected to the substructure and by virtue of the interaction of the connecting device of the patient transfer arrangement and the connecting device of the medical treatment apparatus is fastenable to the medical treatment apparatus in a state detached from the substructure.

4. Patient transfer arrangement according to claim 1,
characterised by a guiding device that includes a guiding element fastened to the carrier member and a guiding element fastened to the substructure and complementary to the guiding element of the carrier member and that, by virtue of the interaction of the guiding element of the carrier member and the guiding element of the substructure, when the carrier member is being detached from the substructure is adapted to enable a guided motion of the carrier member relative to the substructure.

5. Patient transfer arrangement according to claim 1,
characterised in that a guiding element provided on the patient transfer arrangement is adapted to interact with a complementary guiding element provided on the medical treatment apparatus, in order to enable a guided motion of the carrier member relative to the medical treatment apparatus by virtue of the interaction of the guiding element of the patient transfer arrangement and the guiding element of the medical treatment apparatus.

6. Patient transfer arrangement according to claim 1,
characterised by a coupling device that is connectable to a positioning system provided on the medical treatment apparatus for varying the position of the carrier member relative to the medical treatment apparatus.

7. Patient transfer arrangement according to claim 1,
characterised in that the carrier member is adjustable between a first operating position, in which it enables a support of a patient in a sitting position, and a second operating position, in which it enables a support of a patient in a lying position.

8. Patient transfer arrangement according to claim 1,
characterised in that the patient transfer arrangement further includes at least one holding device for fastening a medical support device to the patient transfer arrangement.

9. Medical treatment apparatus with:
a treatment unit,
a base unit, the base unit including a connecting device that is connectable to a complementary connecting device provided on a patient transfer arrangement, in order to fasten a carrier member of the patient transfer arrangement detachably to the medical treatment apparatus in such a position that a patient supported on the carrier member in a sitting or lying position is accessible to a medical treatment by means of the treatment unit of the medical treatment apparatus.

10. Medical treatment apparatus according to claim 9,
characterised in that the medical treatment apparatus is configured in such a way that by virtue of the interaction of the connecting device of the patient transfer arrangement and the connecting device of the medical treatment apparatus the carrier member of the patient transfer arrangement is fastenable to the medical treatment apparatus jointly with a movable substructure of the patient transfer arrangement.

11. Medical treatment apparatus according to claim 9,
characterised in that the medical treatment apparatus is configured in such a way that by virtue of the interaction of the connecting device of the patient transfer arrangement and the connecting device of the medical treatment apparatus the carrier member of the patient transfer arrangement is fastenable to the medical treatment apparatus in a state detached from the substructure of the patient transfer arrangement.

12. Medical treatment apparatus according to claim 9,
characterised by a guiding element that is adapted to interact with a complementary guiding element provided on the patient transfer arrangement, in order to enable a guided motion of the carrier member relative to the medical treatment apparatus by virtue of the interaction of the guiding element of the medical treatment apparatus and the guiding element of the patient transfer arrangement.

13. Medical treatment apparatus according to claim 9,
characterised by a positioning system that is connectable to a coupling device of the patient transfer arrangement and that is adapted to vary a position of the carrier member of the patient transfer arrangement relative to the medical treatment apparatus.

14. Medical treatment apparatus according to claim 13,
characterised by a control unit for controlling the positioning system.

15. Medical treatment apparatus according to claim 9,
characterised in that the treatment unit includes at least one laser source for carrying out refractive surgical interventions on the human eye.

16. Medical treatment system with:
a patient transfer arrangement according to claim 1 and
a medical treatment apparatus according to claim 9.
